(19) United States
(12) Patent Application Publication
(10) Pub. No.: US 2025/0051526 A1
(43) Pub. Date: Feb. 13, 2025

(54) POLYMERS COMPRISING GLYCOLIPIDS AND METHODS FOR PRODUCING AND USING THE SAME

(71) Applicants: ARIZONA BOARD OF REGENTS ON BEHALF OF THE UNIVERSITY OF ARIZONA, Tucson, AZ (US); GlycoSurf, Inc., Salt Lake City, UT (US)

(72) Inventors: Douglas A. Loy, Tucson, AZ (US); David Hogan, Tucson, AZ (US); Raina M. Maier, Tucson, AZ (US); Alicja Babst-Kostecka, Tucson, AZ (US); Susan Perez, Tucson, AZ (US); Chett Boxley, Park City, UT (US); Ryan Stolley, Salt Lake City, UT (US)

(21) Appl. No.: 18/719,173

(22) PCT Filed: Dec. 13, 2022
(86) PCT No.: PCT/US2022/081464

§ 371 Date: Jun. 12, 2024

Related U.S. Application Data
(60) Provisional application No. 63/289,108, filed on Dec. 13, 2021.

(51) Int. Cl. C08J 3/075; C02F 1/56; C02F 101/20; C07H 15/04

(52) U.S. Cl. CPC C08J 3/075; C02F 1/56; C07H 15/04; C02F 2101/20; C08J 2333/04

(57) ABSTRACT

The present invention is directed to polymers comprising glycolipids and methods for producing and using the same. In particular, the polymers of the invention comprise a polymer backbone and a plurality of glycolipid side chains. The glycolipid moieties present in polymers of the invention are metal ion complexing agents. As such, polymers described herein can be used for the remediation of aqueous solutions to remove metal ions.

### CROSS-REFERENCES TO RELATED APPLICATIONS

This application claims benefit of U.S. Provisional Application No. 63/289,108 filed Dec. 13, 2021, the specification of which is incorporated herein in their entirety by reference.

### STATEMENT REGARDING FEDERALLY SPONSORED RESEARCH OR DEVELOPMENT

This invention was made with government support under Grant Nos. R44 ES031897 and P42 ES004940, awarded by National Institutes of Health. The government has certain rights in the invention.

### FIELD OF THE INVENTION

The present invention relates to polymers comprising glycolipids and methods for producing and using the same. In some embodiments, glycolipid moieties present in polymers of the invention are metal ion complexing agents. As such, polymers of the present invention can be used for the remediation of aqueous solutions to remove metal ions.

### BACKGROUND OF THE INVENTION

Metal contamination is present in effluents from mining operations, industrial processes, landfills, wastewater treatment facilities, and both naturally and anthropogenically contaminated groundwaters. Remediation of these waste streams is traditionally accomplished through a variety of technologies: chemical precipitation with hydroxides or sulfides, chelating precipitation, ion exchange, adsorption (using a variety of natural and synthetic adsorbents), membrane filtration, reverse osmosis, electrodialysis, coagulation/flocculation, electrochemical treatment, and flotation. The technology chosen highly depends on the effluent type, and each technology has intrinsic advantages and drawbacks (FIG. 1). For example, chemical precipitation is simple, and capital investments are inexpensive. Still, it is inefficient for low metal concentrations, is non-selective, and generates large amounts of sludge that require subsequent treatment. Membrane filtration technologies, however, have high metal removal efficiencies and generate minimal waste, but they are costly to operate, have high operational complexity, and suffer from membrane fouling.

Accordingly, there is a continuing need for water treatment technology with high efficiency, high treatment capacity, low cost, low waste generation, high reliability, and minimal environmental impact.

### BRIEF SUMMARY OF THE INVENTION

It is an objective of the present invention to provide compositions and methods that allow for useful effluent treatment technology with high efficiency, high treatment capacity, low cost, low waste generation, high reliability, and minimal environmental impact, as specified in the independent claims. Embodiments of the invention are given in the dependent claims. Embodiments of the present invention can be freely combined with each other if they are not mutually exclusive.

Acrylate based hydrogels are environmentally compatible and are characterized by well-defined three-dimensional structures that are porous and contain chemically responsive functional groups. Examples of such chemically responsive functional groups are hydroxyl, thiols, carboxylic acids, amines, phosphinates, and sulfonates. These characteristics are modifiable to target specific contaminants, impart various mechanical characteristics, and enable recovery of captured contaminants through changes in solution chemistry. A hydrogel is a crosslinked hydrophilic polymer that does not dissolve in water but may swell or expand in water. They are highly absorbent yet typically maintain well-defined structures.

Some aspects of the invention provide low cost polymers that are highly efficient in separating metal ions from aqueous solution. Moreover, the polymers of the present invention are highly reliable, have high treatment capacity (i.e., can treat large volumes and achieve metal loading on mass metal per mass gel basis), and have a minimal environmental impact. Furthermore, because the polymers described herein are biodegradable and produced using green chemistry, water treatment using said polymers results in significantly less waste generation than conventional effluent treatment technologies.

In some embodiments, the present invention features polymers comprising a polymer backbone and a plurality of side chains that are covalently linked to said polymer backbone. The polymerization of a monomeric moiety produces the polymer backbone. In some embodiments, the monomeric moiety comprises an acrylate moiety, a methacrylate moiety, a styrenic moiety, or a combination thereof. The polymer backbone may be cross-linked within its framework to provide further cross-linking rather than a simple linear polymer chain. Each of the plurality of side chains includes a glycolipid. The structure or chemical formula of glycolipids may be the same, or the polymer may have two or more different glycolipids as side chains. In some embodiments, the glycolipid comprises a carbohydrate moiety covalently linked to one or more C₄-C₂₀ carboxylate moieties.

In some embodiments, the present invention features methods for removing metal ions from an aqueous solution. The methods described herein may comprise treating an aqueous sample with a polymer comprising a plurality of glycolipid side chains capable of coordinating or forming a complex with the metal ions present in an aqueous sample. As can be appreciated, in addition to the remediation of aqueous samples, methods of the invention can be used to recover valuable metals from aqueous samples. In some embodiments, the metal ion-glycolipid complex that is formed can be removed or separated from the treated aqueous sample to provide at least a partially purified aqueous sample and an isolated metal ion-glycolipid complex. As can be appreciated, in addition to the remediation of aqueous samples, methods of the invention can be used to recover valuable metals from various aqueous samples.

One of the unique and inventive technical features of the present invention is the use of a polymer covalently linked to a glycolipid. Without wishing to limit the invention to any theory or mechanism, it is believed that the technical feature of the present invention advantageously provides for a water treatment technology with high efficiency, high treatment capacity, low cost, low waste generation, high reliability, and minimal environmental impact. None of the presently known prior references or work has the unique, inventive technical feature of the present invention.

Furthermore, the prior references teach away from the present invention. For example, prior art teaches that there would be a regiochemistry issue with regard to the attachment of a glycolipid to a polymer in that the binding site would be blocked. Surprisingly, the present invention achieves a composition in which the binding site is still active.

Furthermore, the inventive technical features of the present invention contributed to a surprising result. For example, it appears that in the attachment of the glycolipid, there is a single site that accounts for the majority of attachment. We would have expected multiple sites of attachment of the acrylate groups.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 is a table outlining some of the advantages and disadvantages of various effluent treatment technologies for removing metal-ion from the mining wastewater stream.

FIGS. 2A, 2B, and 2C show results for glycolipids to chelate metal ions in separate trials (FIGS. 2A, 2B, and 2C), where bars represent the mean and standard deviation of triplicate experiments.

FIG. 3 shows one particular method of modifying a rhamnolipid with an acrylamide group to produce the polymer or hydrogel of the invention by copolymerization of the acrylamide-modified rhamnolipid with a crosslinker to afford a hydrogel having an affinity for metal ions.

FIG. 4 shows one embodiment of the invention for producing selective chelating glycolipid polymers.

FIG. 5 shows one embodiment of the invention for attaching glycolipid to a pre-formed polymer.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

When referring to glycolipids of the invention, the term “derivative” refers to any chemical modification of the parent compound or a compound derived from the parent compound. For example, a derivative of a carbohydrate includes an alkylated carbohydrate, replacement of one or more hydroxyl groups with hydrogen, halide, amine, or a thiol; modification of a hydroxyl group (e.g., by esterification, etherification, protection, etc.); as well as other derivatives known to one skilled in the art. The term carbohydrate includes pyranose and furanose carbohydrates. Exemplary derivatives of carbohydrates include, but are not limited to, alkylated or carboxylated carbohydrates (e.g., one or more hydroxyl groups that are methylated, ethylated, acetylated, or benzoylated), thiol carbohydrate (where one or more hydroxyl groups are replaced with —SH moiety), deoxy carbohydrates (where one or more —OH groups of the carbohydrate are replaced with —H), amine carbohydrates (where one or more —OH groups of the carbohydrate are replaced with —NRaRb, where each of Ra and Rb is independently H, C₁-C₆ alkyl, or a nitrogen protecting group, etc. More specifically, when referring to a carbohydrate, the term “derivative thereof” refers to a derivative of a carbohydrate in which one or more of the hydroxyl groups is replaced with hydrogen (e.g., 2-deoxy glucose, 5-deoxyglucose, etc.), an amine (e.g., amino sugars), a thiol (—SH) or a halogen, such as chloro, fluoro or iodo, (e.g., 5-fluoroglucose, 2-fluoroglucose, 5-chrologlucose, 2-chloroglucose, etc.). In addition, each of the monosaccharides can be an (L)-isomer or a (D)-isomer. The term “a thiol derivative” of a sugar refers to a sugar moiety in which the hydroxyl group that links the “B” moiety in compound of Formula I is replaced with a sulfur atom. (i.e., the linkage between A and B moieties in compound of Formula I is sulfur). Similarly, the term “an amine or amino derivative” of a sugar refers to a sugar moiety in which the hydroxyl group that links the “B” moiety in the compound of Formula I is replaced with a nitrogen atom (i.e., the linkage between A and B is achieved by —NH— moiety).

The term “sugar” and “carbohydrate” are used interchangeably herein and generally refers to a mono- or disaccharide or mixtures thereof. Exemplary carbohydrate that can be used in methods of the invention include, but not limited to, the following carbohydrates:

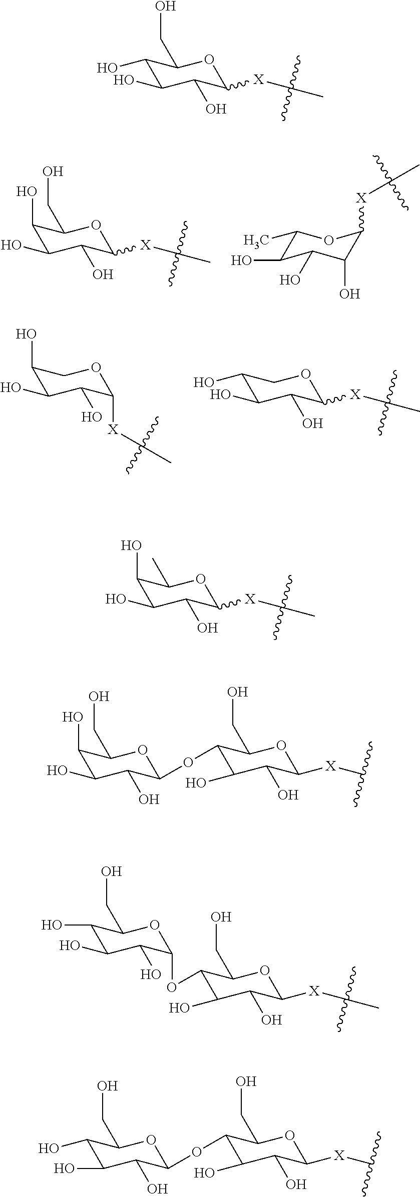

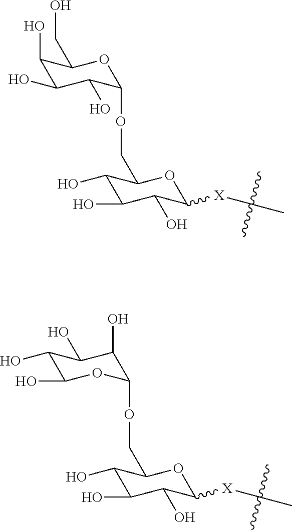

where X is O or S, and where one or more —OH is replaced with H, halogen, or —OR, where R is C₁₋₆ alkyl.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Exemplary monosaccharides of the invention include, but are not limited to, allose, altrose, arabinose, fructose, galactose, glucose, gulose, idose, Ixyose, psicose, rhamnose, ribose, ribulose, sorbose, tagatose, talose, xylose, xylulose, and derivative thereof. Each monosaccharide can also be independently an (L)-isomer or a (D)-isomer.

The term “disaccharide” refers to a carbohydrate composed of two monosaccharides. It is formed when two monosaccharides are covalently linked to form a dimer. The linkage can be a (1→4) bond, a (1→6) bond, a (1→2) bond, a (1→3) bond, etc. between the two monosaccharides. In addition, each of the monosaccharides can be independently an α- or β-anomer. Exemplary disaccharides that can be used in the present invention include, but are not limited to, cellobiose, chitobiose, dirhamnose, gentiobiose, isomaltose, isomaltulose, lactose, lactulose, laminaribose, leucrose, maltose, maltulose, melibiose, nigerose, sophorose, sucrose, terhalose, turanose, xylobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Each monosaccharide can also be independently an (L)-isomer or a (D)-isomer.

Polymers described herein comprise a polymer backbone and a plurality of glycolipid side chains linked (e.g., covalently linked) to the polymer backbone. The term “polymer backbone” refers to the portion of the structure formed by polymerizing monomeric moieties or compounds. The polymer backbone may be a linear polymer or may be cross-linked to form a complex three-dimensional structure.

In some embodiments, the polymer backbone is cross-linked. Unless the context requires otherwise, the terms “side chain” and “side chain glycolipid” may be used interchangeably herein and refer to glycolipid moiety attached to the polymer backbone. As used herein, the term “heavy metal ion” includes transition metal ions and rare earth metal ions, such as lanthanide ions and actinide ions. For the sake of clarity and brevity, methods of the invention will now be described for removing heavy metals. However, it should be appreciated that methods of the invention can be used to remove any metal ion.

Throughout this disclosure, unless the context requires otherwise, the terms “metal ion” and “metal” are used interchangeably herein. When referring to removing metal ions from an aqueous solution, one skilled in the art simply refers to it as removing metal. As such, “removal of metal” is a term of art used by one skilled in the art to refer to removing metal ions. The term “purifying” refers to removing or separating at least a portion of the metal ion(s) that are present in an aqueous sample. In this manner, methods of the invention can be used to treat or remediate effluent water from any natural and/or anthropogenic contaminated water, such as effluents from mining operations, industrial processes, landfills, wastewater treatment facilities, and underground water treatment.

As used herein, the terms “heavy metal” and “metal” are used interchangeably herein and include transition metals and rare earth metals, such as lanthanides and actinides. In one particular embodiment, metal ions are rare earth metal ions (e.g., ions of Y, Ce, Pr, Nd, Pm, Sm, Eu, Gd, Tb, Dy, Ho, Er, Tm, Yb, Lu, La), valuable metal ions (e.g., ions of Cu, Ag, Au, Pd, Pt), and metal ions of environmental concern (e.g., ions of Pb, Cd, Zn, TI, Hg).

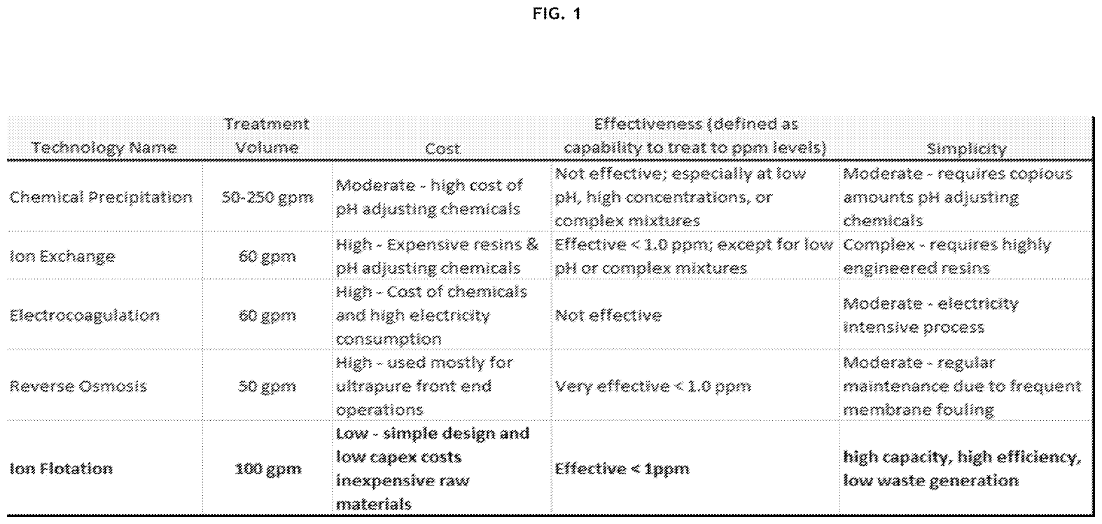

Referring now to FIGS. 1-5, the present invention features compositions and methods for producing the polymers and methods for using the polymers in removing metal ions from an aqueous solution.

The present invention may feature polymers comprising a polymer backbone and a plurality of side chains covalently linked to said polymer backbone. The polymerization of a monomeric moiety may produce the polymer backbone. In some embodiments, the monomeric moiety comprises an acrylate moiety, a methacrylate moiety, a styrenic moiety, or a combination thereof. Each of the plurality of side chains includes a glycolipid. The structure or chemical formula of glycolipids may be the same, or the polymer may have two or more different glycolipids as side chains. In some embodiments, the glycolipid comprises a carbohydrate moiety covalently linked to one or more C₄-C₂₀ carboxylate moieties.

The polymer backbone may be cross-linked within its framework to provide further cross-linking rather than a simple linear polymer chain. In some embodiments, the polymer backbone may be linear. In other embodiments, the polymer backbone may be crosslinked. Crosslinking may be accomplished with monomers bearing two or more polymerizable groups such as acrylates, methacrylates, acrylamides, methacrylamides, and styrenics.

In some embodiments, the polymer is a hydrogel. Yet, in other embodiments, the polymer is a resin.

In some embodiments, the carbohydrate comprises a monosaccharide or a disaccharide, or a combination thereof. Exemplary carbohydrates that can be used include, but are not limited to, glucose, galactose, rhamnose, arabinose, xylose, fucose, lactose, maltose, glucose, fructose, melibiose, cellobiose, rutinose, a thiol derivative thereof, an amine derivative thereof, or a combination thereof.

In some embodiments, the glycolipid comprises the formula:

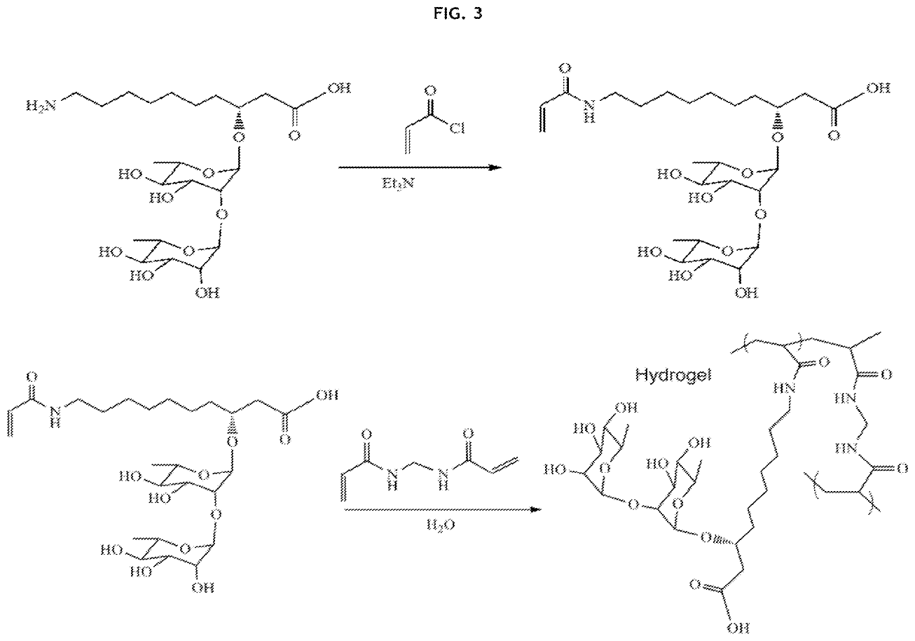

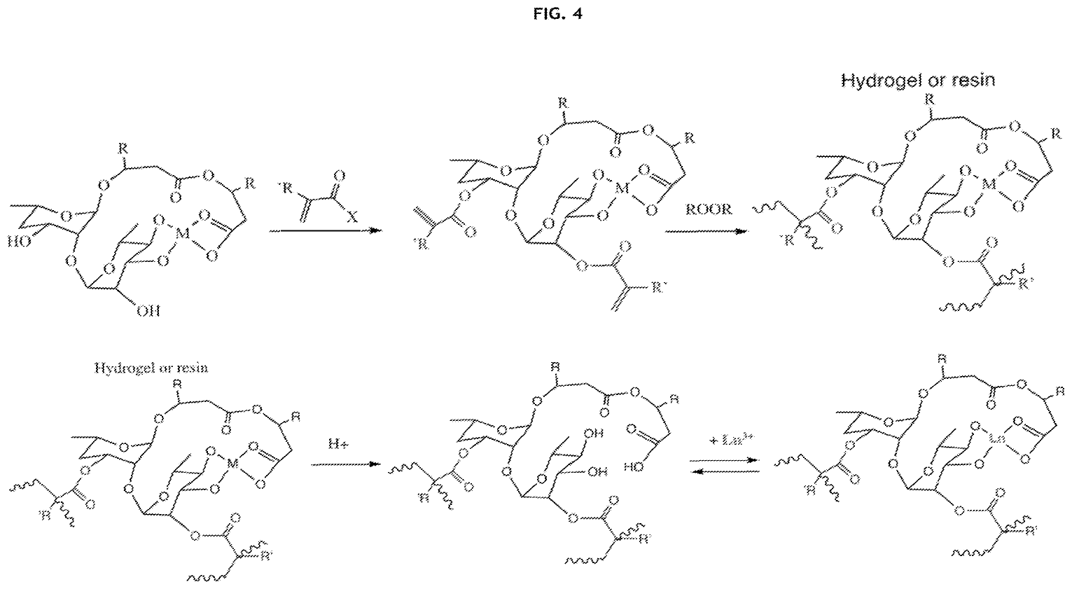

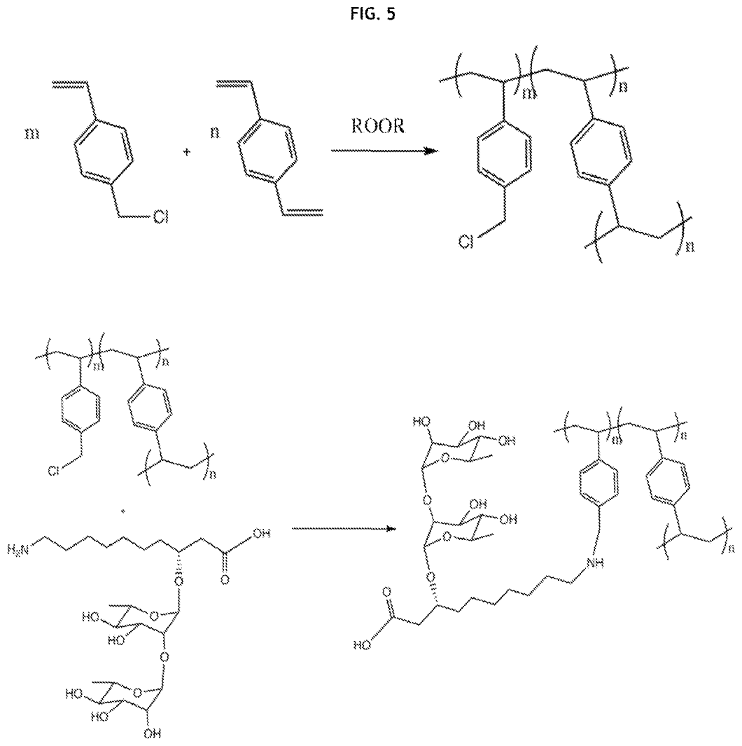

A-[B]ₐ (Formula I)
wherein a is 1 or 2, and wherein A is selected from the group consisting of a monosaccharide, a disaccharide, and a derivative thereof; and each B is attached to a different position of A and is independently a C₈-C₂₀ alkyl or a moiety of the formula:

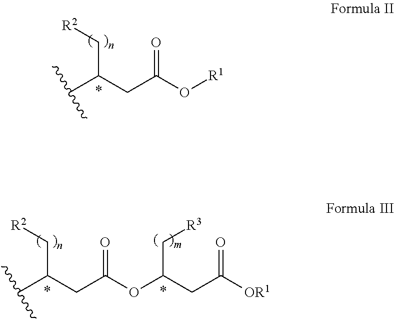

wherein * is a chiral center; each of m and n is independently an integer from 2 to about 20; R¹ is H or C₁-C₂₀ alkyl; each of R² and R³ is independently —CH₃ or —NR⁴-Q; wherein R⁴ is H, C₁-C₈ alkyl, or a nitrogen protecting group; and Q is said polymer backbone, provided said glycolipid is attached to a polymer backbone either through A or B. Connections from A to the polymer backbone would be through ester linkages to carbohydrate hydroxyl moieties (FIG. 4). Connections from B to the polymer backbone through the NR⁴-Q are through either amide linkages to acrylamide monomers (FIG. 3) or amino linkages to styrenic monomers (FIG. 5). In some embodiments, 2 to 3 sites or positions of the glycolipid moiety are attached to the polymer backbone.

The above-mentioned Formula II has one chiral center, whereas Formula III has two chiral centers. While not necessary, one can use enantiomerically enriched moieties of Formula II or Formula III. In general, for cost considerations, a racemic mixture of Formula II or Formula III may be used.

In some embodiments, a is 1. In other embodiments, a is 2. In some embodiments, if a is 1, B (i.e., the fatty acid or lipid moiety) is attached to the anomeric carbon on A (i.e., the sugar moiety). Alternatively, if a is 2, B (i.e., the fatty acid or lipid moiety) may be attached at multiple sites on A (i.e., the sugar moiety), typically a hydroxyl on A or a thiol/amine derivative of A.

In some embodiments, each of m and n is independently an integer from 2 to 20.

In some embodiments, n is an integer from 1 to 30, or 1 to 25, or 1 to 20, or 1 to 15, or 1 to 10, or 1 to 6, or 1 to 5, or 1 to 2, or 2 to 30, or 2 to 25, or 2 to 20, or 2 to 15, or 2 to 10, or 2 to 6, or 2 to 5, or 5 to 30, or 5 to 25, or 5 to 20, or 5 to 15, or 5 to 10, or 5 to 6, or 6 to 30, or 6 to 25, or 6 to 20, or 6 to 15, or 6 to 10, or 10 to 30, or 10 to 25, or 10 to 20, or 10 to 15, or 15 to 30, or 15 to 25, or 15 to 20, or 20 to 30, or 20 to 25, or 25 to 30. Typically n is an integer from 2 to about 25, often from 3 to about 25, and most often from 6 to about 20. Still, in other particular embodiments, n is independently 2, 4, 6, 8, 10, 12, 14, 16, 18, or 20.

In some embodiments, m is an integer from 1 to 30, or 1 to 25, or 1 to 20, or 1 to 15, or 1 to 10, or 1 to 6, or 1 to 5, or 1 to 2, or 2 to 30, or 2 to 25, or 2 to 20, or 2 to 15, or 2 to 10, or 2 to 6, or 2 to 5, or 5 to 30, or 5 to 25, or 5 to 20, or 5 to 15, or 5 to 10, or 5 to 6, or 6 to 30, or 6 to 25, or 6 to 20, or 6 to 15, or 6 to 10, or 10 to 30, or 10 to 25, or 10 to 20, or 10 to 15, or 15 to 30, or 15 to 25, or 15 to 20, or 20 to 30, or 20 to 25, or 25 to 30. Typically m is an integer from 2 to about 25, often from 3 to about 25, and most often from 6 to about 20. Still, in other particular embodiments, m is independently 2, 4, 6, 8, 10, 12, 14, 16, 18, or 20.

In some embodiments, R¹ comprises a hydrogen (H). In other embodiments, R¹ comprises an alkyl group (e.g., a straight-chain alkyl group). For example, R¹ may comprise an alkyl group comprising 1 to 20 carbons. In some embodiments, R¹ may comprise an alkyl group comprising about 1 to 20 carbons, or about 1 to 15 carbons, or about 1 to 10 carbons, or about 1 to 5 carbons, or about 5 to 20 carbons, or about 5 to 15 carbons, or about 5 to 10 carbons, or about 10 to 20 carbons, or about 10 to 15 carbons, or about 15 to 20 carbons.

In some embodiments, R¹ is a straight-chain alkyl group; and not a branch alkyl group. Without wishing to limit the present invention to any theory or mechanism, it is believed that a branch alkyl group may increase toxicity and limit biodegradability.

In some embodiments, R² comprises a methyl group (—CH₃). In some embodiments, R² comprises an amine group (—NR⁴). In some embodiments, R² comprises a (—NR⁴-Q). In some embodiments, R³ comprises a methyl group (—CH₃). In some embodiments, R³ comprises an amine group (—NR⁴). In some embodiments, R³ comprises a (—NR⁴-Q). In some embodiments, R⁴ comprises a hydrogen (H). In some embodiments, R⁴ comprises an alkyl group comprising 1 to 8 carbons. In some embodiments, R⁴ comprises an alkyl group comprising about 1 to 10 carbons, or about 1 to 8 carbons, or about 1 to 6 carbons, or about 1 to 4 carbons, or about 1 to 2 carbons, or about 2 to 10 carbons, or about 2 to 8 carbons, or about 2 to 6 carbons, or about 2 to 4 carbons, or about 4 to 10 carbons, or about 4 to 8 carbons, or about 4 to 6 carbons, or about 6 to 10 carbons, or about 6 to 8 carbons, or about or about 8 to 10 carbons. In other embodiments, R⁴ comprises a nitrogen protecting group. Examples of nitrogen protecting groups include, but are not limited to, benzyl, benzoyl, and tert-butyloxycarbonyl (t-Boc) groups.

In some embodiments, the glycolipid is enantiomerically or diastereomerically enriched. Typically, the carbohydrate portion is enantiomerically enriched as generally biologically derived or produced carbohydrate is used. Yet, in other embodiments, a racemic mixture of the lipid portion or B moiety is used. As can be appreciated, the use of an enantiomerically enriched carbohydrate and a racemic mixture of B moiety results in a diastereomeric mixture of glycolipids.

In some embodiments, A is a sugar comprising a monosaccharide or a disaccharide. In some embodiments, A is a monosaccharide or a thiol derivative thereof, or an amine derivative thereof. Non-limiting examples of monosaccharides may include but are not limited to glucose, galactose, rhamnose, arabinose, xylose, fructose, or fucose.

In some embodiments, B is attached to the hydroxyl group of the anomeric carbon or a thiol derivative thereof of or an amine derivative thereof of said monosaccharide

In other embodiments, A is a disaccharide, or a thiol derivative thereof or an amine derivative thereof. The disaccharide used herein may comprise a 1-2, 1-4, or 1-6 linkage between two monosaccharides. Non-limiting examples of disaccharides may include but are not limited to lactose, maltose, melibiose, cellobiose, sucrose, or rutinose.

The present invention may also feature a composition for producing a polymer, wherein said composition comprises: a glycolipid that is covalently linked to one or more monomeric compounds, wherein each of said monomeric compound is independently an acrylate, a methacrylate, or a styrenic compound. In some embodiments, the glycolipid comprises a monosaccharide or a disaccharide. In some embodiments, the glycolipid comprises a carbohydrate moiety covalently linked to one or more C₄-C₂₀ carboxylate moieties. Exemplary monosaccharides and disaccharides that are useful in the invention include, but are not limited to, glucose, galactose, rhamnose, arabinose, xylose, fucose, lactose, maltose, sucrose, glucose, fructose, melibiose, cellobiose, rutinose, a thiol derivative thereof, and an amine derivative thereof.

In some embodiments, a polymer backbone may be produced by the polymerization of more than 100 monomeric compounds. For example, a polymer backbone may be produced by the polymerization of about 100 monomeric compounds, or about 200 monomeric compounds, or about 300 monomeric compounds, or about 400 monomeric compounds, or about 500 monomeric compounds. In other embodiments, a polymer backbone may be produced by the polymerization of more than 500 monomeric compounds.

In other embodiments, the glycolipid is of the formula:

A-[B]ₐ
a is an integer from 1 to 2; A is selected from the group consisting of a monosaccharide, a disaccharide, and a derivative thereof; and each B is attached to a different position of A and is independently a C₈-C₂₀ alkyl or a moiety of the formula:

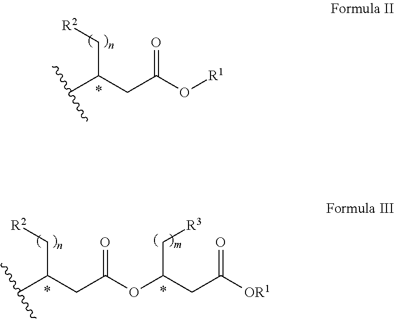

wherein * is a chiral center; each of m and n is independently an integer from 2 to about 20, typically from 2 to about 25, often from 3 to about 25, and most often from 6 to about 20; R¹ is H or C₁-C₂₀ alkyl; each of R² and R³ is independently —CH₃ or —NR⁴-Q; R⁴ is H, C₁-C₈ alkyl, or a nitrogen protecting group; and Q is a polymerizable group that said glycolipid is attached to by at least one of B or A.

In some embodiments, the glycolipid is covalently linked to one monomeric moiety. In other embodiments, the glycolipid is covalently linked to two monomeric moieties. In some embodiments, the glycolipid is covalently linked to three monomeric moieties. In some embodiments, the glycolipid is covalently linked to four monomeric moieties In some embodiments, the glycolipid is covalently linked to five monomeric moieties. In some embodiments, each hydroxyl on the sugar moiety may be modified with polymerizable groups. For example, there are three in the case of monorhamnolipid and five in the case of dirhamnolipid. The number of sites will vary based on the sugar moiety A.

The present invention may further feature methods for purifying an aqueous solution by removing a metal ion from an aqueous solution comprising said metal ion. The method may comprise contacting an aqueous solution with a polymer as described herein (e.g., a polymer comprising a polymer backbone and a plurality of glycolipid side chains linked to said polymer backbone) under conditions sufficient to form a metal-ion glycolipid complex and separating said metal-ion glycolipid complex from said aqueous solution to produce at least a partially purified aqueous solution. In some embodiments, the steps of the method may be repeated.

In some embodiments, contacting an aqueous solution with a polymer comprises passing an aqueous solution (e.g., a metal-bearing solution) through a polymer-packed column. In other embodiments, contacting an aqueous solution with a polymer comprises adding a polymer (e.g., as a gel or beads) to a bulk aqueous solution containing metal cations. In the latter example, the polymer would be removed from the aqueous solution once the reaction was complete.

In some embodiments, conditions that may affect the formation of the metal ion-glycolipid complex include but are not limited to pH, ratio of metal ion concentration to glycolipid binding sites, metal speciation, type of glycolipid used, and other matrix constituents.

In some embodiments, separating said metal-ion glycolipid complex from the aqueous solution comprises any physical separation that allows the fluid phase to be decanted from the polymer, including but not limited to: skimming, filtration, or settling.

The metal ion-glycolipid complex that is formed can be removed or separated from the treated aqueous sample to provide at least a partially purified aqueous solution. In general, any metal ion that can coordinate with glycolipid moiety can be removed from the aqueous sample. In some embodiments, methods of the invention remove heavy metal ions.

In some embodiments, the aqueous solution may comprise water (e.g., contaminated water, such as effluents from mining operations, industrial processes, landfills, wastewater treatment facilities, and underground water treatment). In some embodiments, the aqueous solution may be solutions produced from oil and gas production (e.g., water), geothermal brines, water bodies with naturally high metal ion concentrations, radionuclide-contaminated water in nuclear energy/weapons facilities.

In some embodiments, the metal ion is selected from the group consisting of an ion of U, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, TI, Hg, or a combination thereof. However, it should be appreciated that the scope of the invention is not limited to these metal ions. In general, any metal ions can be removed by methods of the invention.

In some embodiments, the polymer is a hydrogel. There are a variety of technologies in which hydrogels can be utilized for metal recovery. For example, gels can be packed into columns through which a solution containing the metal cations is passed. The metal cations exchange into the gel's chelation sites, and the gel eventually saturates with metal. The metal ions can be released by any one of the various methods known to one skilled in the art including, but not limited to, switching the flow from the metal solution to an aqueous acid solution that, due to many orders of magnitude higher concentration, exchange places with the metal cation in a concentrated solution. Once the metal has been exchanged for protons, the column can be used to extract additional metals. The process of chelating and releasing metal ions can be automated using a central processing unit and an appropriate detector, such as a mass spectrometer, infrared or UV-Vis detector, etc.

Chelated metal ions can also be released from the hydrogel by dispersing the hydrogel in foam to allow the hydrogel to be used in running or standing water in the field. The metal cations exchange with protons into the gel. Periodically, the metal cations are released by removing the foam containing the hydrogel from the water and immersing it in a tank with aqueous acid. As with the column, the hydrogel-foam composites can be used, recycled, and reused to remove metal from ponds or other water streams.

In some embodiments, the hydrogels described herein may be formulated as spherical hydrogels. This spherical hydrogel can be placed in an aqueous sample and used as a metal binding or chelating agent. The hydrogel spheres can be added dry or swollen with water to water containing metal cations. In some embodiments, the dry hydrogels absorb water and expand as much as 100×. Metal in the water binds to the chelating sites. Without being bound by any theory, it is believed the glycolipids that are attached to the polymer backbone are the active sites for metal ion binding. Pre-swollen hydrogels can also be used instead of dry hydrogels. In both cases, the hydrogels of the invention will float in the water with neutral buoyancy and can be collected with a net or screen or simply filtered. As described above, the metal ions can be released by immersing the hydrogel spheres in acidic water. The acid-treated hydrogels can be recycled and reused.

The strength of the hydrogels can be tailor made by changing the weight percent polymer in the hydrogel composition. In general, higher-weight percent gels are stronger and will withstand more physical damage. In general, any known methods for using hydrogels in water remediation or metal ion removal can be used. Accordingly, exemplary applications disclosed herein are provided solely for the purpose of illustrating methods of the invention and are not intended to limit the scope of the invention.

The polymers disclosed herein are unique in that they are combined with glycolipid molecules alone or in combination with a templating technology (e.g., the glycolipid may be reacted with a specific metal ion prior to creating the polymer; thus creating a metal-harvesting polymer that is tuned to the metal ion used to template the glycolipid). In some embodiments, hydrogels can be formulated or tailored to have either broad or narrow selective affinity for metals. Specifying the selectivity range allows the capture of entire metal groups, e.g., rare earth elements, or specific individual metals, e.g., a single targeted rare earth element. In some embodiments, hydrogels of the invention use various glycolipids modified by polymerizable acrylate or methacrylate groups. The glycolipids included in this disclosure are generally a group of bioinspired glycolipids consisting of various hydrophilic mono- and disaccharide moieties-including but not limited to rhamnose, xylose, galactose, cellobiose, lactose—and hydrophobic moiety varying with regard to lipid chain length, number, and saturation. These glycolipids are based on the structure of the extensively studied rhamnolipid biosurfactants.

Rhamnolipids exhibit high surface activity (e.g., reduce surface and interfacial tensions), and are traditionally derived from biological sources. They are considered “green” molecules due to their natural products from bacteria (e.g., Pseudomonas aeruginosa), biodegradability, and low toxicity. The present inventors have experimentally determined the monorhamnolipid-metal conditional stability constants (log 3) for 26 metals of interest. These data show that monorhamnolipid (mRL) exhibits a binding preference for metals of environmental concern (e.g., Cd, Pd, Hg) over common soil and water cations by 6-8 orders of magnitude.

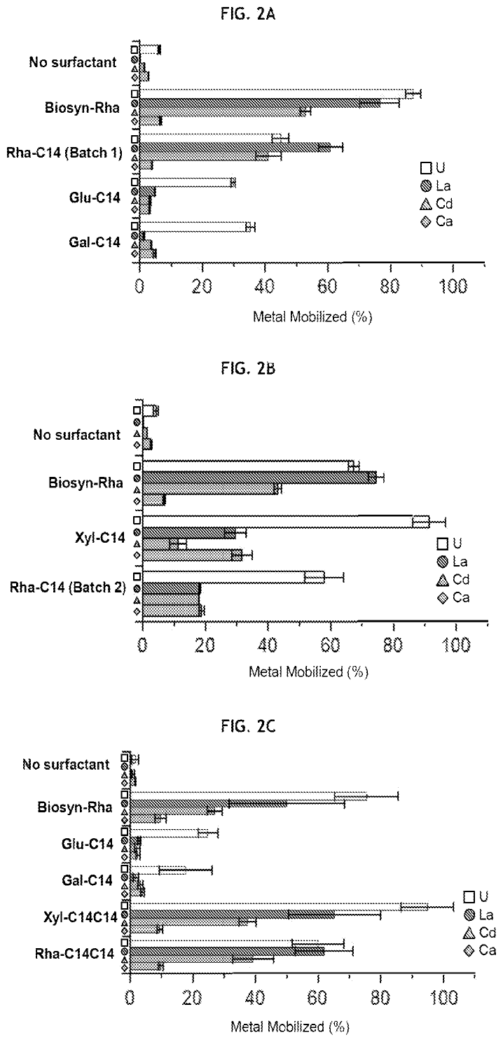

Prior to incorporation into the polymeric structure, metal binding capability of glycolipids were determined. In these experiments, the metals UO₂²⁺ (log β=9.82), La³⁺ (log β=9.29), Cd²⁺ (log β=6.89), and Ca²⁺ (log β=4.10) were adsorbed onto a cation binding resin that was placed in a small container with 10 ml of water. The glycolipids were then introduced into the container and allowed to mix with the resin. After mixing, it was determined that glycolipids successfully bound, i.e., chelated, the metal. The degree of removal is indicative of the strength of interaction as the reaction is a competition between the cation binding resin and the glycolipid. FIG. 2 shows the results of three independent experiments demonstrating the ability of various glycolipids to mobilize metals from the cation binding resin.

Methods of the invention include combining, admixing, or contacting an aqueous solution of a polymer of the invention with an aqueous sample under conditions sufficient to form glycolipid-metal ion complex. The resulting polymer comprising glycolipid-metal ion complex is then separated from the mixture by any of the separation methods known to one skilled in the art, such as, but not limited to, centrifugation, filtration, settling, etc. Separated metals can then be recovered by any of the methods known to one skilled in the art including, but not limited to, treating the separated polymer with acidic solution, etc.

In some embodiments, the polymer is a resin. In this manner, a simple filtration of the resin can be used to remove the metal ion glycolipid complex.

Glycolipids of the invention can be readily prepared using, for example, procedures disclosed in commonly assigned U.S. patent application Ser. No. 15/358,159, which is incorporated herein by reference in its entirety.

### Example

The following is a non-limiting example of the present invention. It is to be understood that said example is not intended to limit the present invention in any way. Equivalents or substitutes are within the scope of the present invention.

Two synthetic approaches for producing polymers comprising glycolipid side chains are illustrated below. However, it should be appreciated that one skilled in the art, having read the present disclosure, can readily apply other known methods to produce polymers comprising glycolipids.

Broad specificity polymers via modification of amine-terminated lipid in glycolipids with acrylamide group: When recovering metals from dilute aqueous solutions, polymers with broad specificity are useful to generate initial metal concentrates of target metals, e.g., the rare earth elements. Concentrates generated with this polymer can then be processed through metal-specific adsorbents to separate individual metals. The synthetic approaches for these materials are outlined below using the rhamnolipid structure as an illustrative example in the schemes. However, it should be appreciated that other glycolipids may be used to produce a wide variety of polymers, such as hydrogels.

The amine group is substantially more reactive than the alcohol hydroxy groups allowing single-point functionalization of the rhamnolipid with a polymerizable acrylamide group. Copolymerization of the monomer with a crosslinker (FIG. 3) in water yields a hydrogel with high levels of metal binding rhamnolipid. This limits that the polymerizable group (i.e., monomer or crosslinking group) will not interfere with the metal complexation process.

Narrow specificity gels via templating hydrogels and ion exchange resins with glycolipids: Rare earth cation complexes modified with polymerizable groups: Pre-complexation of the rhamnolipid with a lanthanide cation before modifying with a polymerizable acrylate group prevents the modification from interfering with the metal complexation, and may provide a mechanism for templating resins or polymers made from the monomer to have greater selectivity for rare earth metals (FIG. 4). Once the monomer is copolymerized into a network polymer, acid exchange liberates the site with the hydroxyl and carboxylate groups prepositioned to coordinate rare earth metal cations.

In addition to the hydrogels, the preparation of a solid-state resin functionalized with chelating rhamnolipid can also be used via the following approach. Free radical polymerization of chloromethyl styrene with a small amount of divinylbenzene allows very high loadings of rhamnolipid to be attached to the solid resin for rare earth metal complexation (FIG. 5). Generally, about 1% to 2% of chloromethylstyrene is used in the resins. Referring again to FIG. 5, amine groups react with the chloromethyl group to install groups for solid-state synthesis; these resins require low levels of functionality to achieve controlled syntheses. A cation exchange resin for rare earth elements does not require dilution of the functionality and can benefit from having higher levels of functionalization.

In general, any monomers or crosslinking compounds that can be attached to glycolipids and polymerized in solution (e.g., water, alcohol, organic solvent, or a mixture thereof) can be used in producing polymers of the present invention. In some embodiments, polymers of the present invention are produced using comonomer(s), as illustrated in FIG. 2. to provide copolymerized glycolipids. In such methods, a polymerizable monomer is attached to the glycolipid, and the resulting modified glycolipid is polymerized with comonomer compounds.

Exemplary metals that can be separated from a sample include, but are not limited to, rare earth metals (such as Y, Ce, Pr, Nd, Pm, Sm, Eu, Gd, Tb, Dy, Ho, Er, Tm, Yb, Lu, La), valuable metals (such as Cu, Ag, Au, Pd, Pt), and metals of environmental concern (such as Pb, Cd, Zn, TI, Hg).

In the forthcoming examples, procedures that are constructively reduced to practice are described in the present tense, and procedures that have been carried out in the laboratory are set forth in the past tense.

Example 1: Rhamnolipids are modified with polymerizable acrylate or methacrylate groups for preparation of hydrogels with enhanced metal affinities. The rhamnolipids hydroxyl groups are modified using acryloyl chloride or methacrylic anhydride. There are five hydroxyl groups on the rhamnolipid that can be modified. Some of these are required in the coordination of the metal cations that give rise to the molecule's metal affinity. In order to control the regioselectivity of the acrylate attachment and to avoid reduction in metal binding, the rhamnolipid are templated around a rare earth cation (see FIG. 4) to direct the acylation reaction to the two hydroxy groups not required for metal coordination. Modification of the rhamnolipid with a single acrylate or methacrylate group affords a monomer that can be polymerized to afford linear, soluble polymers. Attachment of two groups afford a crosslinker monomer that can be copolymerized with the linear monomer to afford the crosslinked network needed for hydrogel formation. Thus, all of the monomers in the hydrogel can be modified with the metal-binding rhamnolipid leading to the maximum possible metal capacity. Once the rhamnolipid is polymerized in aqueous solution to a hydrogel, the metal cation can be exchanged with protons by washing the hydrogel with acid. The resulting hydrogel can be characterized structurally and mechanically

Example 2: Metal coordination is evaluated using hydrogels produced in Example 1, and greenhouse studies are performed to identify the suitable volume and shape of the hydrogel to enhance the uptake and allocation of target elements to above-ground plant tissues. Metals tested include zinc and copper, as well as selected Rare Earth Elements (REE). Metal/REE uptake by hydrogels is determined using a known methodology. For hydrogel-plant studies, a plant collection is generated and maintained consisting of 3 model plant species with known abilities to accumulate high concentrations of desired metals in their foliage. Plant growth and development, as well as metal/REE uptake by gels and plants, are monitored in a controlled growth chamber in hydroponics.

Plant cuttings are transferred into 1 L polyethylene pots (one to four plants per pot). Each pot is filled with a slightly modified half-strength nutrient solution with 20 μM FeEDDHA, 500 μM MgSO4, 1 mM NH4H2PO4, 0.1 μM (NH4)6Mo7O24, 0.1 μM CuSO4, 25 μM H3BO3, 2 μM MnSO4, 1 μM KCl, 0.1 μM NaCl, 2 mM Ca(NO3)2, 3 mM KNO3, and 1 μM Zn—added as ZnSO4—within a 2 mM 2-morpholinoethanesulphonic acid (MES) buffer, set to pH 5.5 using KOH. Metal/REE is added and the solution is changed weekly. The experimental setup includes: a no-plant control and 3 plants species×3 volumes of hydrogel (see Objective 1)×10 replicates, and a no-hydrogel control with plants×10 replicates=150 total pots. Every seven days, the following steps are performed: i) measure the photosynthetic efficiency of the foliage; ii) take above- and below-ground digital images; and iii) estimate total leaf area based on digital photographs and Easy Leaf Area software. Additionally, small leaf samples are harvested along with subsamples of hydrogels to monitor metal/REE uptake into the plant material and in the hydrogel.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A polymer comprising a polymer backbone and a plurality of side-chains covalently linked to said polymer backbone, wherein (i) the polymer backbone is produced by polymerization of a monomeric moiety comprising an acrylate moiety, a methacrylate moiety, a styrenic moiety, or a combination thereof, and (ii) each of said plurality of side-chains comprise a glycolipid, wherein the glycolipid comprises a carbohydrate moiety covalently linked to one or more C₄-C₂₀ carboxylate moieties.

2. The polymer of claim 1, wherein said polymer backbone is linear.

3. The polymer of claim 1, wherein said polymer backbone is cross-linked.

4. The polymer of claim 1, wherein said polymer is a hydrogel.

5. The polymer of claim 1, wherein said polymer is a resin.

6. The polymer of claim 1, wherein said carbohydrate comprises a monosaccharide or a disaccharide, or a combination thereof.

7. The polymer of claim 6, wherein said carbohydrate comprises glucose, galactose, rhamnose, arabinose, xylose, fucose, lactose, maltose, glucose, fructose, melibiose, cellobiose, rutinose, a thiol derivative thereof, an amine derivative thereof, or a combination thereof.

8. The polymer of claim 1, wherein said glycolipid is a moiety of the formula:
A-[B]ₙ
wherein a is an integer from 1 to 2; A is selected from the group consisting of a monosaccharide, a disaccharide, and a derivative thereof; and each B is attached to a different position of A and is independently a C₆-C₂₀ alkyl or a moiety of the formula: 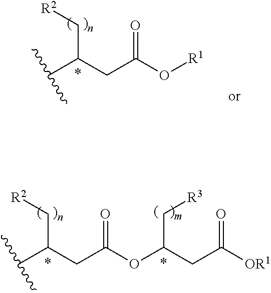
wherein * is a chiral center; each of m and n is independently an integer from 2 to 20; R¹ is H or C₁-C₂₀ alkyl; each of R² and R³ is independently —CH₃ or —NR⁴-Q; R⁴ is H, C₁-C₈ alkyl, or a nitrogen protecting group; and Q is a polymerizable group that said glycolipid is attached to by at least one of B or A.

9. A composition for producing a polymer, wherein said composition comprises: a glycolipid that is covalently linked to one or more monomeric compounds, wherein each of said monomeric compound is independently an acrylate, a methacrylate, or a styrenic compound.

10. The composition of claim 9, wherein said glycolipid is covalently linked to one to five monomeric compounds.

11. The composition of claim 9, wherein said glycolipid comprises a saccharide selected from the group consisting of glucose, galactose, rhamnose, arabinose, xylose, fucose, lactose, maltose, glucose, fructose, melibiose, cellobiose, rutinose, a thiol derivative thereof, and an amine derivative thereof.

12. The composition of claim 9, wherein said glycolipid is of the formula:
A-[B]ₐ
wherein a is an integer from 1 to 2; A is selected from the group consisting of a monosaccharide, a disaccharide, and a derivative thereof; and each B is attached to a different position of A and is independently a C₆-C₂₀ alkyl or a moiety of the formula: 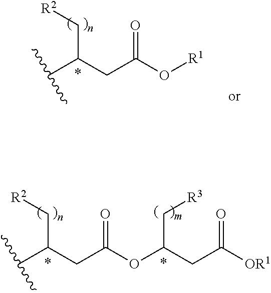
wherein * is a chiral center; each of m and n is independently an integer from 2 to 20; R¹ is H or C₁-C₂₀ alkyl; each of R² and R³ is independently —CH₃ or —NR⁴-Q; R⁴ is H, C, —C₈ alkyl, or a nitrogen protecting group; and Q is a polymerizable group that said glycolipid is attached to by at least one of B or A.

13. The composition of claim 9, wherein said glycolipid comprises at least two monomeric compounds.

14. A method for purifying an aqueous solution by removing a metal ion from an aqueous solution containing said metal ion, said method comprising:
a. contacting an aqueous solution with a polymer comprising a polymer backbone and a plurality of glycolipid side-chains that are covalently linked to said polymer backbone under conditions sufficient to form a metal-ion glycolipid complex; and
b. separating said metal-ion glycolipid complex from said aqueous solution to produce at least a partially purified aqueous solution,
wherein said polymer backbone is produced by polymerization of a monomeric moiety comprising an acrylate moiety, a methacrylate moiety, a styrenic moiety, or a combination thereof, and (ii) each of said plurality of glycolipid side-chains comprises a carbohydrate moiety that is covalently linked to one or more C₄-C₂₀ carboxylate moieties.

15. The method of claim 14, further comprising repeating steps (a) and (b).

16. The method of claim 14, wherein said metal ion is selected from the group consisting of an ion of uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, TI, Hg, or a combination thereof.

17. The method of claim 14, wherein said polymer is a hydrogel.

18. The method of claim 14, wherein said polymer is a resin.

19. The method of claim 14, wherein each of said plurality of glycolipid side-chain is independently of the formula:
A-[B]ₙ
wherein a is an integer from 1 to 2; A is selected from the group consisting of a monosaccharide, a disaccharide, and a derivative thereof; and each B is attached to a different position of A and is independently a C₆-C₂₀ alkyl or a moiety of the formula: 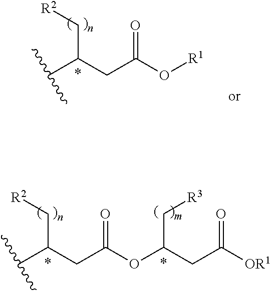
wherein * is a chiral center; each of m and n is independently an integer from 2 to 20; R¹ is H or C₁-C₂₀ alkyl; each of R² and R³ is independently —CH₃ or —NR⁴-Q; R⁴ is H, C₁-C₈ alkyl, or a nitrogen protecting group; and Q is the polymerizable group that said glycolipid is attached to by at least one of B or A.
